(19) United States
(12) Patent Application Publication
(10) Pub. No.: US 2025/0302433 A1
(43) Pub. Date: Oct. 2, 2025

(54) ULTRASOUND PROBE

(71) Applicant: FUJIFILM Corporation, Tokyo (JP)

(72) Inventor: Koichi ISHIDA, Tokyo (JP)

(73) Assignee: FUJIFILM Corporation, Tokyo (JP)

(21) Appl. No.: 19/095,578

(22) Filed: Mar. 31, 2025

(30) Foreign Application Priority Data
Apr. 1, 2024 (JP) 2024-059049

(51) Int. Cl. A61B 8/12; A61B 8/00; A61B 90/00

(52) U.S. Cl. CPC A61B 8/12; A61B 8/4444; A61B 90/39; A61B 2090/3937

(57) ABSTRACT

An ultrasound probe inserted into a body cavity includes: an ultrasound transducer that radiates an ultrasound wave; and a case that accommodates the ultrasound transducer. The case includes a first puncture guide that is a hole or a notch into which a puncture needle is inserted and that guides a puncture direction of the puncture needle, and a guide marker that is provided on an outer surface of the case and that indicates a guide direction of the puncture guide.

### CROSS REFERENCE TO RELATED APPLICATION

This application claims priority to Japanese Patent Application No. 2024-059049 filed on Apr. 1, 2024, which is incorporated herein by reference in their entireties including the specifications, claims, drawings, and abstracts.

### BACKGROUND OF THE INVENTION

### 1. Field of the Invention

The present specification discloses an ultrasound probe that is inserted into a body cavity.

### 2. Description of the Related Art

In the related art, a technique has been known in which an ultrasound probe is inserted into a body cavity and ultrasound diagnosis is performed in order to know an internal state of an organ of a subject (for example, a patient or an animal). In the ultrasound diagnosis, the ultrasound probe is brought into contact with a surface of the organ. In this state, ultrasound waves are transmitted from the ultrasound probe to the inside of the organ, and reflected waves thereof are received. Then, an ultrasound tomographic image indicating the internal state of the organ is formed based on the obtained reflected wave signal. An operator, such as a doctor, performs various treatments, for example, collection of cells or injection of a drug by a puncture needle on the inside of the organ, while referring to the obtained ultrasound tomographic image.

Here, in the related art, an ultrasound probe having a puncture guide that guides a traveling direction of a puncture needle is known. For example, WO2015/166302A discloses an ultrasound probe that is inserted into a body cavity and that has a puncture guide. In WO2015/166302A, the puncture guide is a hole or a groove that extends in a guide direction of the puncture needle. The puncture needle is stably guided in the traveling direction by the puncture guide.

### SUMMARY OF THE INVENTION

However, in the ultrasound probe according to the related art, it is difficult to know the traveling direction of the hole or the groove which is the puncture guide. As a result, a problem with the related art is that it is difficult for an operator to know the guide direction by the puncture guide.

In addition, in the case of a puncture probe used outside the body, a large puncture guide is attached to the side of the ultrasound probe. Therefore, the operator can see the shape of the large puncture guide to easily know the guide direction. In the case of an ultrasound probe for a body cavity, it is not possible to attach the large puncture guide, and it is difficult for the operator to know the guide direction.

Therefore, the present specification discloses an ultrasound probe that enables a user to easily know a guide direction by a puncture guide.

According to an aspect of the present invention, there is provided an ultrasound probe that is inserted into a body cavity and that comprises: an ultrasound transducer that radiates an ultrasound wave; and a case that accommodates the ultrasound transducer. The case includes a puncture guide that is a hole or a notch into which a puncture needle is inserted and that guides a puncture direction of the puncture needle, and a guide marker that is provided on an outer surface of the case and that indicates a guide direction of the puncture guide.

The provision of the guide marker enables the operator to easily know the guide direction by the puncture guide.

In this case, the guide marker may be a figure that indicates an inclination of a wall in an end portion of the puncture guide in an axial direction.

This configuration enables the operator to intuitively know the guide direction by the puncture guide.

In addition, the puncture guide may have a tapered portion whose dimension in a front-rear direction decreases as the tapered portion advances from an entrance into the case, and the guide marker may have a substantially triangular shape that is surrounded by a first line indicating an inclination of a wall at one end of the puncture guide in the axial direction, a second line indicating an inclination of a wall at the other end of the puncture guide in the axial direction, and a third line connecting the first line and the second line.

In this configuration, since the guide marker is large and conspicuous, the operator can easily know the guide direction. In addition, the operator can intuitively know an insertable range of the puncture needle only by referring to one guide marker.

Further, the guide marker may include a line that indicates a trajectory of a central axis of the puncture needle advancing along the wall in the end portion of the puncture guide in the axial direction.

This configuration enables the operator to easily estimate the movement trajectory of the central axis of the puncture needle and thus to perform a more appropriate puncture treatment.

The color of the guide marker may be opposite to a color of the case.

This configuration enables the operator to more clearly recognize the guide marker.

According to the technology disclosed in the present specification, the operator can easily know the guide direction by the puncture guide.

### BRIEF DESCRIPTION OF THE DRAWINGS

FIG. 1 is a schematic view showing an aspect of use of an ultrasound probe.

FIG. 2 is a perspective view showing a distal end portion of the ultrasound probe.

FIG. 3 is a side view showing the distal end portion of the ultrasound probe and shows an ultrasound tomographic image.

FIG. 4 is a cross-sectional view taken along line A-A of FIG. 3.

FIG. 5 is a cross-sectional view taken along line B-B of FIG. 4.

FIG. 6 is a schematic view showing a relationship among a guide hole, a puncture needle, and a guide marker.

FIG. 7 is a schematic view showing a relationship between the guide hole and the guide marker.

FIG. 8 is a view showing an example of another ultrasound probe.

FIG. 9 is an image diagram showing a relationship between a radiation direction of an ultrasound wave and a target part.

### DESCRIPTION OF THE PREFERRED EMBODIMENTS

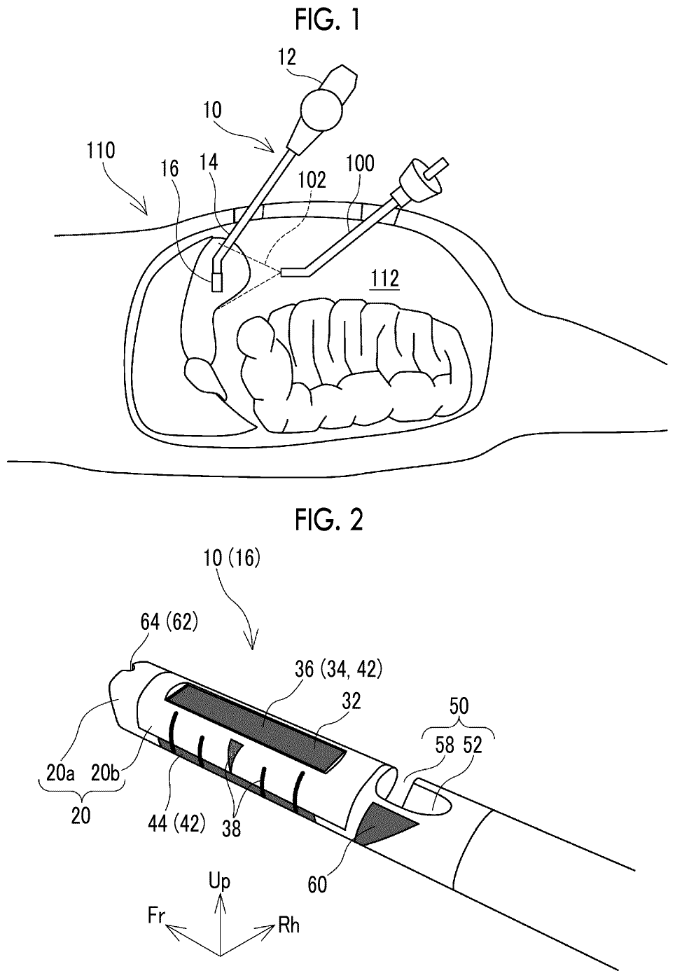

Hereinafter, a configuration of an ultrasound probe 10 will be described with reference to the drawings. FIG. 1 is a schematic view showing an aspect of use of the ultrasound probe 10. In addition, hereinafter, a laparo-probe used in an abdominal cavity will be described as an example. However, the technology disclosed in the present specification is not limited to the laparo-probe as long as it is inserted into a subject 110, and other types of ultrasound probes 10 may be used. In addition, the subject may be a person or an animal.

The ultrasound probe 10 according to the present example is used in laparoscopic surgery. In the laparoscopic surgery, an operator inserts an endoscope 100, the ultrasound probe 10, and other surgical instruments (for example, forceps, an electric scalpel, and the like) (not shown) from a port into an abdominal cavity 112. A camera is provided in the endoscope 100, and a video captured by the camera is displayed on a display (not shown) in real time. The operator operates the ultrasound probe 10 or other surgical instruments while observing the video on the display.

In addition, the operator knows an internal state of an organ (for example, a liver) in a body cavity using the ultrasound probe 10. Then, the operator performs a predetermined treatment (for example, puncture or the like) on the organ based on the obtained information.

The ultrasound probe 10 is roughly divided into an operation portion 12, an insertion portion 14, and a distal end portion 16. The operation portion 12 is a portion held by the operator. The operation portion 12 is provided with a plurality of controls (for example, buttons, dials, and the like) that receive various operations.

The insertion portion 14 is a tubular member that is inserted into the subject. The insertion portion 14 can be bent by operating the operation portion 12, and the position and orientation of the distal end portion 16 are changed by bending the insertion portion 14. A signal cable for transmitting and receiving an electric signal and a transmission cable for transmitting a force for bending the insertion portion 14 are provided in the insertion portion 14.

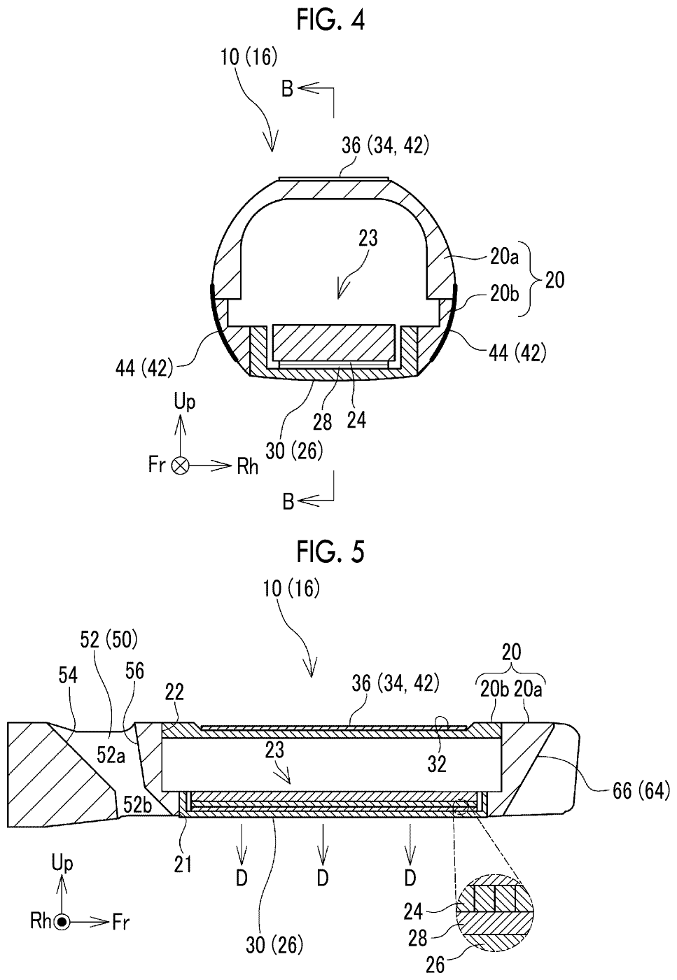

The distal end portion 16 is attached to a terminal of the insertion portion 14. The distal end portion 16 has an ultrasound transducer 23 (not shown in FIG. 1, see FIGS. 4 and 5) and transmits and receives ultrasound waves. The distal end portion 16 will be described with reference to FIGS. 2 to 5.

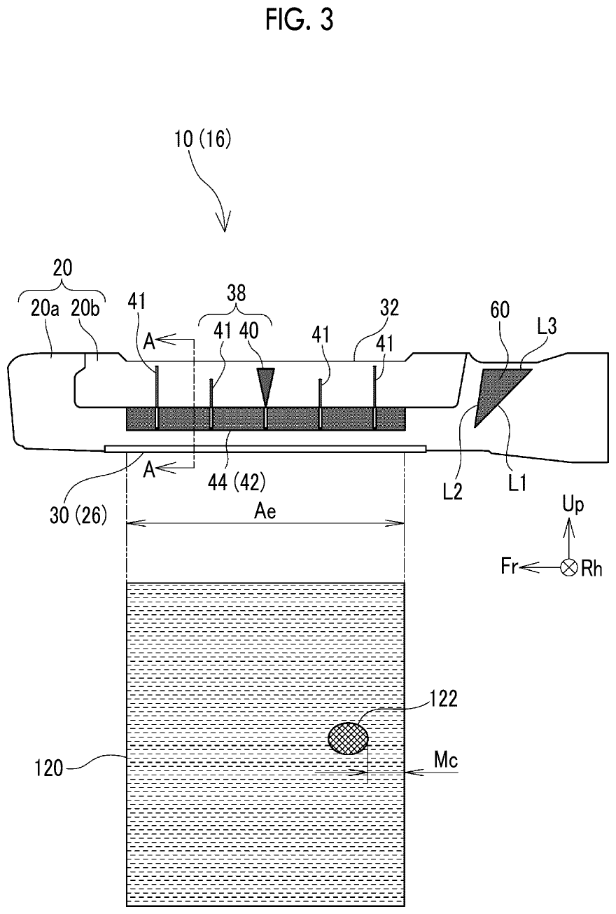

FIG. 2 is a perspective view showing the distal end portion 16. In addition, FIG. 3 is a side view showing the distal end portion 16 and is an image diagram showing an ultrasound tomographic image 120 obtained by the ultrasound probe 10. Further, FIG. 4 is a cross-sectional view taken along line A-A of FIG. 3, and FIG. 5 is a cross-sectional view taken along line B-B of FIG. 4. Furthermore, hereinafter, in order to clarify directions, an axial direction of a case 20 is referred to as a “front-rear direction”, a direction parallel to a radiation direction D of the ultrasound waves is referred to as an “up-down direction”, and a direction orthogonal to the front-rear direction and the up-down direction is defined as a “left-right direction”.

The distal end portion 16 has the case 20 that has a substantially cylindrical shape and the ultrasound transducer 23 that is accommodated in the case 20. The ultrasound transducer 23 has a plurality of transducer elements 24 (see FIG. 5) that transmit and receive the ultrasound waves. Further, in FIG. 5, the transducer element 24 is shown to be larger or smaller than the actual size for ease of understanding. The ultrasound probe 10 according to the present example is a linear probe that performs linear scanning with an ultrasound beam, and the plurality of transducer elements 24 are linearly arranged in the front-rear direction (that is, the axial direction of the case 20). A matching layer 28 and an acoustic lens 26 are disposed in a thickness direction of the transducer element 24. The ultrasound beam passes through the matching layer 28 and the acoustic lens 26 and is radiated to the outside of the ultrasound probe 10. Therefore, an outer surface of the acoustic lens 26 is a radiation surface 30 for transmitting and receiving the ultrasound waves.

In a case where the ultrasound waves are transmitted from the ultrasound transducer 23 to an object, the transmitted ultrasound waves are sequentially reflected from an acoustic impedance discontinuous surface of the object. The ultrasound transducer 23 receives the reflected waves and converts the reflected waves into an electric signal (that is, a reflected wave signal). The reflected wave signal is transmitted to an ultrasound diagnostic apparatus (not shown) at any time. The ultrasound diagnostic apparatus generates the ultrasound tomographic image 120 (see FIG. 3) of the object based on the reflected wave signal output from the ultrasound probe 10.

The ultrasound transducer 23 is accommodated in the case 20. As described above and shown in FIGS. 2 and 4, the case 20 has a substantially cylindrical shape. In the present example, the case 20 has a first case piece 20a and a second case piece 20b. The first case piece 20a is a substantially cylindrical member in which a portion of a peripheral surface is chipped. A lens hole 21 (see FIG. 5) to which the acoustic lens 26 is fitted and an assembly hole 22 (see FIG. 5) to which the second case piece 20b is fitted are formed in the peripheral surface of the first case piece 20a. The assembly hole 22 is a hole that faces the lens hole 21. In a process of manufacturing the ultrasound probe 10, the ultrasound transducer 23 is disposed in the case 20 through the assembly hole 22. The second case piece 20b is attached to the first case piece 20a after the ultrasound transducer 23 and the like are assembled.

However, the configuration of the case 20 is an example and may be appropriately changed. Therefore, the case 20 may be configured by one component or may be configured by three or more components. In addition, in the present example, the acoustic lens 26 is exposed to the outside through the lens hole 21. However, the acoustic lens 26 may be completely accommodated in the case 20. In this case, the lens hole 21 is not formed in the case 20, and the case 20 is made of a material having ultrasound wave transmittance. In addition, in this case, a portion of the case 20 that faces the acoustic lens 26 is the radiation surface 30.

In the case 20, a range bar 44 is provided in a portion that is close to the radiation surface 30 of the ultrasound waves in a circumferential direction. As shown in FIG. 4, a total of two range bars 44 are provided on both sides of the radiation surface 30 in the circumferential direction, respectively. The range bar 44 is a strip-shaped pattern that is elongated in the front-rear direction. The range of the range bar 44 in the front-rear direction is matched with the range of a radiation range Ae of the ultrasound waves in the front-rear direction (see FIG. 3). The range bar 44 is formed by, for example, painting, plating, laser printing, or the like. The range bar 44 functions as a range marker 42 indicating the radiation range Ae of the ultrasound waves. The reason for providing the range marker 42 will be described below.

Further, a scale 38 is provided on the peripheral surface of the case 20. As shown in FIG. 3, the scale 38 has a center mark 40 and a plurality of lines 41 that are arranged at equal intervals from the center mark 40. The center mark 40 is a mark that indicates the center of the radiation range Ae of the ultrasound waves in the axial direction. In the present example, the center mark 40 is an isosceles triangle that faces downward (that is, faces the radiation surface 30). The plurality of lines 41 are arranged at equal intervals on both sides of the center mark 40 in the front-rear direction. Each line 41 extends in the circumferential direction from the inside of the range bar 44 to the outside of the range bar 44. Therefore, it can be said that a portion of the scale 38 overlaps the range marker 42.

In the case 20, a recessed portion 32 that is recessed from a surrounding area is formed on a side that is 180 degrees opposite to the radiation surface 30. A direction plate 36 is disposed in the recessed portion 32. The direction plate 36 is a flat plate having a flat upper surface. A surface of the direction plate 36 is orthogonal to the radiation direction D of the ultrasound waves and functions as the direction marker 34 indicating the radiation direction D of the ultrasound waves, which will be described below.

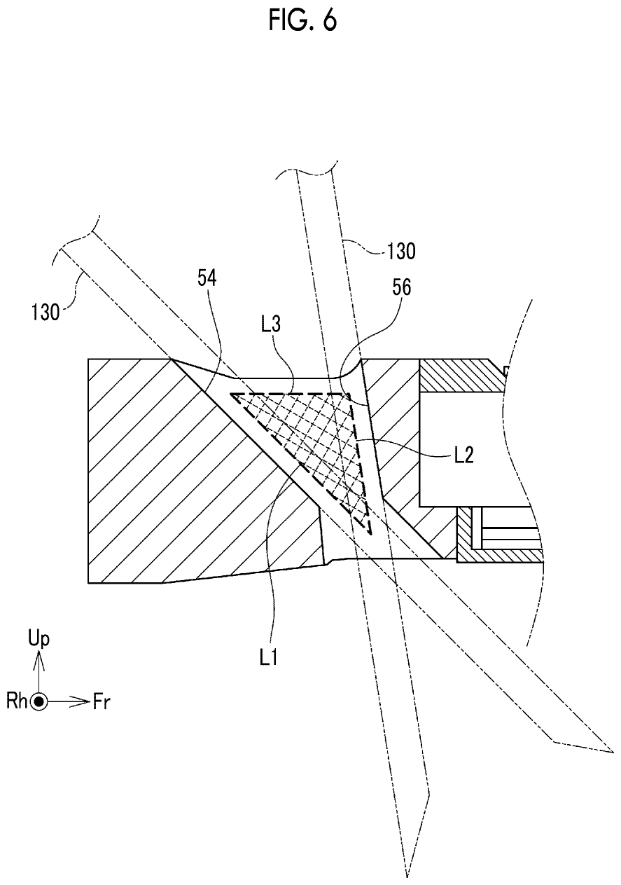

The case 20 is further provided with two puncture guides 50 and 62 (see FIGS. 2 and 5). Both of the puncture guides 50 and 62 are portions that guide a traveling direction of a puncture needle 130 (see FIG. 6). The first puncture guide 50 is disposed closer to a proximal side than the recessed portion 32. The first puncture guide 50 includes a guide hole 52 that penetrates the case 20 in the up-down direction and a lateral hole 58 (see FIG. 2) that connects the guide hole 52 and a side surface of the case 20. As shown in FIG. 5, the guide hole 52 has an hourglass shape whose dimension in the front-rear direction decreases toward the center in the up-down direction. In other words, the guide hole 52 is roughly divided into an upper tapered portion 52a, whose dimension in the front-rear direction decreases as the upper tapered portion 52a advances from an entrance into the case 20, and a lower tapered portion 52b, whose dimension in the front-rear direction decreases as the lower tapered portion 52b advances from an exit into the case 20. Hereinafter, one end surface of the upper tapered portion 52a in the front-rear direction is referred to as a “first wall 54”, and the other end surface thereof in the front-rear direction is referred to as a “second wall 56”. In a case where the operator inserts the puncture needle 130, the operator slides the puncture needle in a state of pressing the puncture needle 130 against the first wall 54 or the second wall 56. Then, the puncture needle 130 advances stably in the direction defined by the first wall 54 or the second wall 56.

The second puncture guide 62 is disposed at a terminal of the case 20. In the present example, the second puncture guide 62 includes a guide groove 64 (see FIGS. 2 and 5) that is formed in a terminal surface of the case 20. As shown in FIG. 5, the guide groove 64 has a third wall 66 that advances to the proximal side as it goes down. In a case where the operator inserts the puncture needle 130, the operator slides the puncture needle 130 in a state of pressing the puncture needle 130 against the third wall 66. Then, the puncture needle 130 advances stably in the direction defined by the third wall 66.

Here, as is clear from FIGS. 2 and 3, in the present example, the guide marker 60 is provided on the side surface of the case 20. The guide marker 60 is a pattern that indicates a guide direction of the puncture needle 130 by the first puncture guide 50. More specifically, the guide marker 60 is a triangle that is surrounded by a first line L1 indicating the inclination of the first wall 54 of the guide hole 52, a second line L2 indicating the inclination of the second wall 56, and a third line L3 connecting the first line L1 and the second line L2.

The reason for providing the guide marker 60 will be described. In general, in a case where the operator punctures the target part 122 inside the organ with the puncture needle 130, the operator estimates the actual position of the target part 122 based on the ultrasound tomographic image 120. Then, the operator adjusts the position of the ultrasound probe 10 (and thus the puncture guide 50 or 62) or adjusts the puncture angle of the puncture needle 130 such that the puncture needle 130 can reach the estimated position of the target part 122. However, in general, the puncture guide is a hole or a groove, and it is difficult for the operator to visually recognize the angle of the wall surface of the puncture guide from the outside. In particular, in a case where the ultrasound probe 10 is present in the body cavity, the operator can check the ultrasound probe 10 only through the image captured by the camera of the endoscope 100. In addition, a visual field 102 of the camera of the endoscope 100 is narrow. Therefore, in a case where the ultrasound probe 10 is present in the body cavity, it is difficult for the operator to accurately know the angle of the wall surface of the puncture guide. As a result, in the related art, since the operator does not know the guide direction of the puncture needle 130 by the puncture guide, the operator may not insert the puncture needle 130 at an appropriate position and angle. In a case where the position or angle of the puncture needle 130 is not appropriate, it is necessary to reinsert the puncture needle 130. In this case, the invasiveness of the puncture treatment is increased.

Therefore, in the ultrasound probe 10 according to the present example, the guide marker 60 indicating the guide direction by the first puncture guide 50 is provided on the side surface of the case 20. As described above, the guide marker 60 is a figure that indicates the inclination angle of the first wall 54 and the inclination angle of the second wall 56 in the guide hole 52. Therefore, the operator can easily know the traveling direction of the puncture needle 130 inserted along the first wall 54 or the second wall 56 with reference to the guide marker 60.

Further, in the puncture treatment, a central axis of the puncture needle 130 needs to reach a target position (for example, the center of the target part 122). Therefore, in the present example, the guide marker 60 is configured such that a trajectory of the central axis of the puncture needle 130 advancing along the first wall 54 or the second wall 56 is known. FIG. 6 is a schematic view showing a relationship among the guide hole 52, the puncture needle 130, and the guide marker 60. As shown in FIG. 6, the first line L1 is a line obtained by projecting the trajectory of the central axis of the puncture needle 130 advancing along the first wall 54 onto the peripheral surface of the case. Similarly, the second line L2 is a line obtained by projecting the trajectory of the central axis of the puncture needle 130 advancing along the second wall 56 onto the peripheral surface of the case. This configuration enables the operator to easily know the movement trajectory of the central axis of the puncture needle 130 with reference to the guide marker 60. Therefore, this makes it easy for the operator to insert the puncture needle 130 such that the central axis of the puncture needle 130 reaches the target position.

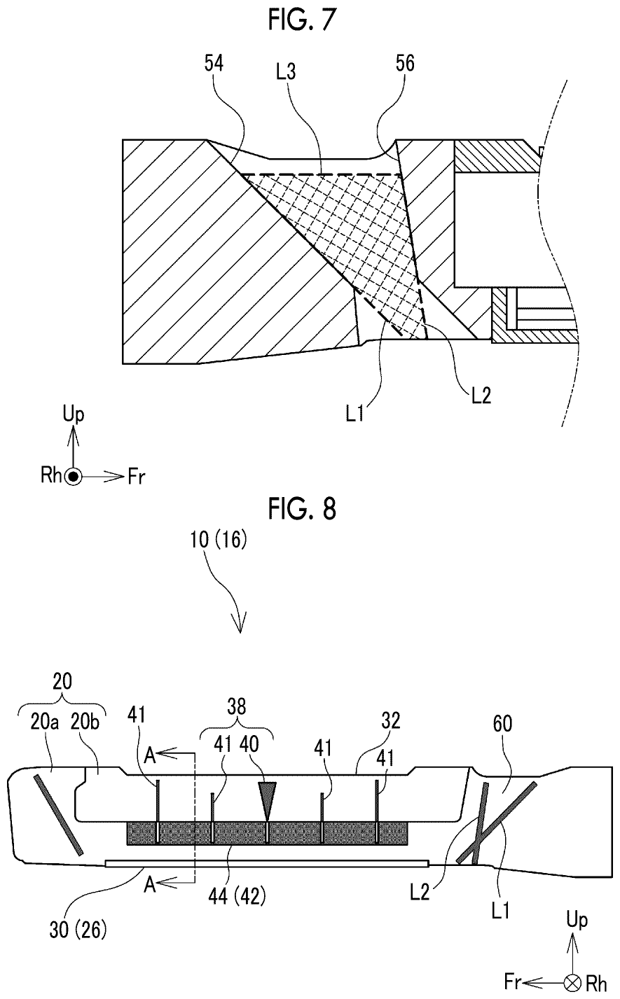

However, this configuration is an example, and the guide marker 60 may have other forms as long as the guide marker 60 indicates the guide direction by the puncture guide. For example, as shown in FIG. 7, the first line L1 and the second line L2 may be lines obtained by projecting the first wall 54 and the second wall 56 onto the peripheral surface of the case 20, respectively. In this configuration, the guide marker 60 has a shape that does not depend on the diameter of the puncture needle 130. Therefore, one guide marker 60 can correspond to a plurality of types of puncture needles 130 having different diameters.

In addition, the guide marker 60 is not limited to a closed shape, such as a triangle, and may be simple lines as shown in FIG. 8. In addition, in the above-described example, the guide marker 60 is provided only beside the guide hole 52 (that is, the first puncture guide 50). However, as shown in FIG. 8, the guide marker 60 may be provided beside the guide groove 64 (that is, the second puncture guide 62). This configuration enables the operator to know the guide direction well even in a case in which the second puncture guide 62 is used.

In addition, as is clear from FIG. 2, the guide marker 60 is slightly separated from the entrance of the guide hole 52, and there is a slight blank region between the guide marker 60 and the guide hole 52. This is to simplify a process of printing the guide marker 60. That is, the guide marker 60 is formed by, for example, laser printing. In the case of the laser printing, printing is not capable of being performed on the entire circumference of the cylindrical case 20 by one printing process. Therefore, in the case of the laser printing, the case 20 is divided into a plurality of areas (for example, three areas) in the circumferential direction in advance, and the printing process is performed in order on each of the plurality of areas. In the present example, the case 20 is divided into three areas in the circumferential direction, and the guide marker 60 is set to a size that fits within one of the three areas. With this configuration, the blank region is formed between the guide marker 60 and the guide hole 52, but the guide marker 60 can be formed by one printing process, which makes it possible to simplify the printing process. In addition, the method for forming the guide marker 60 is not limited to the laser printing, and the guide marker 60 may be formed by another process, for example, plating or painting.

Further, as is clear from the above description, the ultrasound probe 10 according to the present example has the direction plate 36, the range bar 44, and the scale 38. The reason for providing these elements will be described. As described above, the ultrasound probe 10 according to the present example is inserted into the abdominal cavity 112 and is then used. In this case, the position and posture of the ultrasound probe 10 are checked by the camera of the endoscope 100. In addition, the operator knows the internal state of the organ from the ultrasound tomographic image 120 obtained by the ultrasound probe 10.

Here, a case where a predetermined treatment is performed on a target part 122 shown in the ultrasound tomographic image 120 (see FIG. 3) is considered. In this case, the operator estimates the actual position of the target part 122 from the position of the target part 122 in the ultrasound tomographic image 120. In a case where the actual position of the target part 122 is estimated, it is necessary to accurately know the radiation range Ae and the radiation direction D of the ultrasound waves.

For example, in a case where the operator resects the target part 122, the operator specifies a distance from an end portion of the ultrasound tomographic image 120 to the target part 122 as a resection margin Mc. The resection margin Mc corresponds to the actual distance from an end portion of the radiation range Ae of the ultrasound waves to the target part 122. Therefore, in order to know the actual position of the target part 122, it is important to know the end portion of the radiation range Ae of the ultrasound waves.

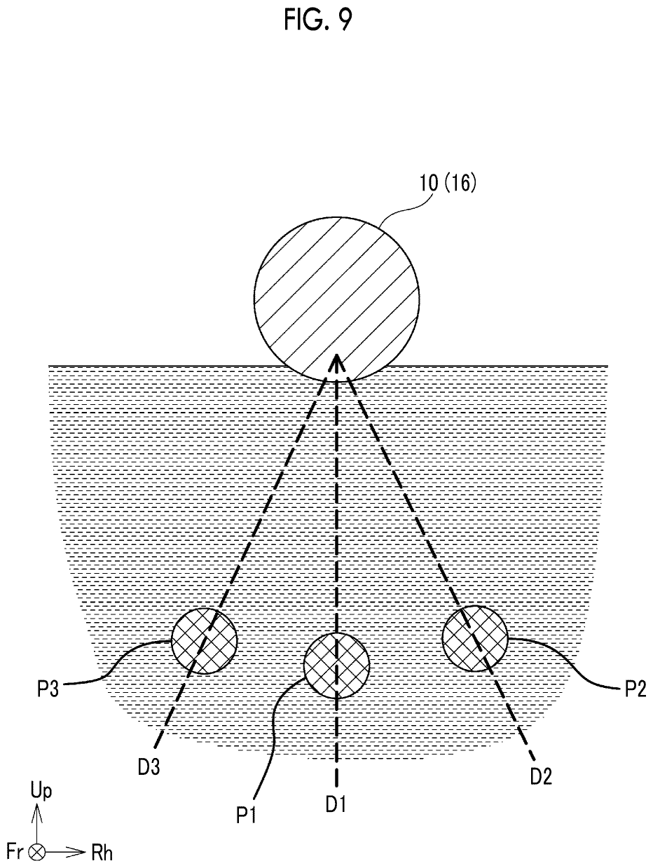

In addition, even in a case where the ultrasound tomographic images 120 are the same, the actual position of the target part 122 differs depending on the radiation direction D of the ultrasound waves. For example, in a case where the radiation direction D of the ultrasound waves is a direction D1 in FIG. 9, it can be estimated that the target part 122 is located at a position P1. Similarly, in a case where the radiation direction D of the ultrasound waves is a direction D2 in FIG. 9, it can be estimated that the target part 122 is located at a position P2. In a case where the radiation direction D of the ultrasound waves is a direction D3 in FIG. 9, it can be estimated that the target part 122 is located at a position P3. As described above, the radiation direction D of the ultrasound waves is very important in estimating the actual position of the target part 122.

Therefore, in the present example, the range bar 44 and the direction plate 36 are provided in the case 20 in order to easily know the radiation range Ae and the radiation direction D. The range of the range bar 44 in the front-rear direction is matched with the range of the radiation range Ae of the ultrasound waves in the front-rear direction, and the range bar 44 functions as the range marker 42 indicating the radiation range Ae. Therefore, the operator can easily know the radiation range Ae with reference to the range bar 44. In addition, the range bar 44 is provided on both sides of the radiation surface 30 in the circumferential direction. Therefore, even in a state in which the radiation surface 30 is pressed against the surface of the organ, the operator can easily visually recognize the range bar 44. Further, since the range bar 44 is provided on both sides of the radiation surface 30, the operator can clearly know the radiation range Ae even in a case where the endoscope 100 is present on either the left or right side of the ultrasound probe 10.

Meanwhile, in order to appropriately treat the target part 122, a mark indicating the end portion of the radiation range Ae may be placed on the surface of the organ. The marking is performed, for example, by burning a very small portion of the surface of the organ. In a case where the range bar 44 is far away from the surface of the organ, a marking position is likely to deviate. In the present example, the range bar 44 is disposed close to the radiation surface 30. Therefore, in a case where the radiation surface 30 is pressed against the surface of the organ, the range bar 44 is naturally close to the surface of the organ. Therefore, this enables the operator to accurately mark the end portion of the radiation range Ae on the surface of the organ.

In addition, as described above, the scale 38 is disposed to partially overlap the range bar 44. Therefore, the operator can clearly know the distance as well as the radiation range Ae. Therefore, this enables the operator to clearly know the actual position of the target part 122.

In addition, as described above, the ultrasound probe 10 is further provided with the direction plate 36. The range of the direction plate 36 in the front-rear direction is matched with the range of the radiation range Ae in the front-rear direction. Therefore, the direction plate 36 also functions as the range marker 42 indicating the radiation range Ae. As a result, it can be said that the ultrasound probe 10 according to the present example has three range markers 42 in the circumferential direction. The interval at which the three range markers 42 are disposed is less than 180 degrees. Therefore, at least one range marker 42 can be visually recognized from any direction in 360 degrees around the case 20. Therefore, even in a case where only a narrow visual field 102 (see FIG. 1) is obtained as in the endoscope 100, the operator can reliably know the range marker 42 and thus the radiation range Ae. For example, the range bar 44 may not be visible to the camera of the endoscope 100 depending on the positional relationship between the endoscope 100 and the ultrasound probe 10. Even in this case, the operator can see the direction plate 36 to clearly know the radiation range Ae.

In addition, an upper surface of the direction plate 36 is orthogonal to the radiation direction D of the ultrasound waves, and the direction plate 36 functions as the direction marker 34 indicating the radiation direction D of the ultrasound waves. Therefore, the operator can observe the direction plate 36 to know the radiation direction D of the ultrasound waves and thus the actual position of the target part 122. In addition, the direction plate 36 is a rectangular shape that is elongated in the front-rear direction. In a case where the direction plate 36 has a simple geometric shape, such as a rectangle, the operator can easily recognize the inclination of the direction plate 36 and thus the radiation direction D of the ultrasound waves from the appearance of the angle of the side or corner portion of the direction plate 36.

In addition, the direction marker 34 may have other forms as long as the radiation direction D of the ultrasound waves can be known. For example, a plane parallel to the radiation direction D of the ultrasound waves may be provided as the direction marker 34 in the case 20. In addition, the number of planes functioning as the direction marker 34 is not limited to one, and a plurality of planes may be provided. For example, the plane orthogonal to the radiation direction D may be disposed in the upper surface of the case 20, and the plane parallel to the radiation direction D may be disposed in the side surface of the case 20.

Meanwhile, all of the colors of the range bar 44, the scale 38, the direction plate 36, and the guide marker 60 are opposite to the color of the case 20. For example, in a case where the color of the case 20 is a light color, such as white or silver, the color of the range bar 44 or the like is a dark color such as black or gray. This coloring makes it possible to improve the visibility of the range bar 44 or the like and makes it possible for the operator to easily recognize the radiation range Ae and radiation direction of the ultrasound waves and the guide direction of the puncture needle.

In addition, the configurations described above are only examples, and other configurations may be changed as appropriate as long as the ultrasound probe 10 has the characteristics described in claim 1. Therefore, the ultrasound probe 10 may not have the range marker 42, the direction marker 34, and the scale 38 as long as the ultrasound probe 10 has the guide marker 60 indicating the guide direction. Furthermore, the ultrasound probe 10 described above is a linear probe in which the plurality of transducer elements 24 are linearly arranged. However, the technology disclosed in the present specification is not limited to the linear probe and may be applied to other types of probes, for example, convex probes.

What is claimed is:

1. An ultrasound probe inserted into a body cavity, the ultrasound probe comprising:
an ultrasound transducer that radiates an ultrasound wave; and
a case that accommodates the ultrasound transducer,
wherein the case includes
a puncture guide that is a hole or a notch into which a puncture needle is inserted and that guides a puncture direction of the puncture needle, and
a guide marker that is provided on an outer surface of the case and that indicates a guide direction of the puncture guide.

2. The ultrasound probe according to claim 1,
wherein the guide marker is a figure that indicates an inclination of a wall in an end portion of the puncture guide in an axial direction.

3. The ultrasound probe according to claim 2,
wherein the puncture guide has a tapered portion whose dimension in a front-rear direction decreases as the tapered portion advances from an entrance into the case, and
the guide marker has a substantially triangular shape that is surrounded by a first line indicating an inclination of a wall at one end of the puncture guide in the axial direction, a second line indicating an inclination of a wall at the other end of the puncture guide in the axial direction, and a third line connecting the first line and the second line.

4. The ultrasound probe according to claim 3,
wherein the guide marker includes a line that indicates a trajectory of a central axis of the puncture needle advancing along the wall in the end portion of the puncture guide in the axial direction.

5. The ultrasound probe according to claim 1,
wherein a color of the guide marker is opposite to a color of the case.
